(19) United States
(12) Patent Application Publication
(10) Pub. No.: US 2022/0395220 A1
(43) Pub. Date: Dec. 15, 2022

(54) TWO-PROCESS SLEEP WHEEL

(71) Applicant: Douglas E. Moul, Willoughby Hills, OH (US)

(72) Inventor: Douglas E. Moul, Willoughby Hills, OH (US)

(21) Appl. No.: 17/344,199

(22) Filed: Jun. 10, 2021

(51) Int. Cl. A61B 5/00

(52) U.S. Cl. CPC A61B 5/4812; A61B 5/743; A61B 5/4809

(57) ABSTRACT

A wheel chart for visualizing sleep pressure on a person's body by manipulating a series of moveable circular disks positioned on a common axis wherein the wheel includes a first disk representing a circadian (C) process and second disk representing a homeostatic (S) process. The disks are rotatable relative to each other to simulate how the C and S processes increase and decrease sleep pressure.

### BACKGROUND

The present exemplary embodiment relates to a wheel chart for showing the physiological effects of the Circadian (C) and Homeostatic (S) Processes on sleep. It finds particular application in conjunction with natural rhythms and will be described with particular reference thereto. However, it is to be appreciated that the present exemplary embodiment is also amenable to other like applications.

Some people suffering from a delayed sleep phase syndrome have a natural impulse to advance the timing of light exposure. Others will also do this to adjust their body's clock before traveling to a new time zone. If done too early, the person risks causing further phase delay or jet lag. In these and other scenarios, a visual aid could help convey the concept of the phase response curve in a way that shows people how their internal (or body) clock can be shifted somewhat independently of the current time. When explaining circadian sleep disorders, it is difficult to help people understand how phase responses work in relation to retinal exposures to light (over a 24-hour period). Doctors also face challenges when informing patients on how melatonin—taken in the evening—may affect a phase advance.

People suffering from insomnia could also benefit from understanding how their native sleep pressures operate.

Sleep health information may help people understand the physiological reasons how certain (e.g., behavioral) changes or cues will affect their sleeping habits. One known visual aid for conveying such information is a graph displayed in a linear format for time. One problem with the linear graph is that it does not show how key processes work in parallel and potentially independently. When thinking about these processes, a key challenge is to understand that these processes act like clocks that can act independently from each other, and even from environmental time. There is no known visual aid or graph that may be used to educate the lay public about the physiological time-anatomy of sleep pressures in a way that conveys these processes as independent clocking mechanisms against each other. Therefore, an improved circadian graph is desired for educating patients and/or medical professionals (in clinical and non-clinical settings) about the overall cyclicity of daily physiological processes. Disclosed herein is a sleep wheel chart that employs polar coordinates to display sleep heath information.

### BRIEF DESCRIPTION

One embodiment of the disclosure relates to a wheel chart including a series of moveable circular disks positioned on a common axis. The disks include a first disk representing a Circadian (C) Process (or Process C) and second disk representing a Homeostatic (S) Process (or Process S). The disks are rotatable relative to each other to show how the C and S Processes interact to effect sleep pressure. By rotating the disks, a user can use the wheel chart to simulate sleep pressure on a person's body by manipulating the interaction between Process C and Process S.

The first disk displays information that can include a Phase Response Curve (PRC); a heat map of Process C sleep pressures; and a region in which combination of the two processes can be jointly appreciated. The PRC is a line graph rendered on a circular axis along which units of time are displayed. In one embodiment, the units of time scaled on the PRC are in increments of hours of anticipated shifts that would result from retinal exposures to strong light, or from ingestion of melatonin at particular times over a 24-hour period. The PRC is graphed throughout the 24-hour period in units of shift in phase advance or phase delay. The units of time in a 24-hour clock display can be displayed on a fixed circular disk on which the first disk overlays.

In one embodiment, the first disk can include the PRC as an outer concentric ring along an edge of the first disk and a first heat map as an inner concentric ring relative to the PRC.

The second disk includes a second heat map, wherein the second heat map translucently overlaps the first heat map in at least a region of the inner concentric ring. The first disk and second disk each include heat map regions that at least partially overlap, so that their joint physiological pressures toward sleep can be visualized when the disks are independently rotated.

A method of visualizing the shifting of a body's circadian rhythm using a wheel chart, comprising a series of moveable circular disks positioned on a common axis, the disks including a first disk representing a circadian (C) process and second disk representing a homeostatic (S) process, wherein the disks are rotatable relative to each other to show how the circadian (C) and homeostatic (S) processes interact to effect sleep pressure, wherein the first disk displays a Phase Response Curve (PRC), the method comprising: selecting a goal wakeup time in relation to a current environmental time; then setting the circadian (C) process disk to the current environmental time; then determining whether the goal wakeup time requires a delay or advance of the body's circadian rhythm: then using the PRC to establish a new wake up time to delay or advance the circadian rhythm; then setting the circadian (C) process disk to the new wake up time; and then repeating the last two steps, if necessary, until the new wake up time is the goal wakeup time.

In one embodiment, the wheel chart can further include a third rotatable disk representing morning light exposure and/or a fourth rotatable representing evening light exposure. In the contemplated embodiment, each rotatable disk includes a tab for rotating the disk around the common axis. All the translucent disks are independently rotatable around the common axis against the backing, which displays a 24-hour clock face.

Another embodiment of the disclosure relates to a wheel chart graphing a Circadian (C) Process and a Homeostatic (S) Process on separate disks. The disks are rotatable relative to one another to display the physiological processes governing sleep and circadian rhythms.

In this embodiment, the circadian process can be graphed as a circular line graph and the Homeostatic (S) Process can be graphed as a circular heat map. Both heat maps are displaced in a near-to-continuous format, to reflect the near-to-continuous processes that occur naturally. The line graph and the heat map can be rotatable relative to one another to display an effect of the combined C and S Processes on sleep pressure. The circular line graph is a Phase Response Curve (PRC) rendered on a circular axis along which units of time are displayed in increments over a 24-hour period of shift in phase advance or phase delay.

The Circadian (C) Process can be graphed as a first circular heat map and the homeostatic (S) process can be graphed as a second circular heat map. The first and second heat maps are rotatable relative to one another and at least partially overlap to display an effect of the combined C and S Processes on sleep pressure.

In one embodiment, the wheel chart includes a PRC as an outer concentric ring on a first disk and at least one heat map as an inner concentric ring relative to the PRC. The at least one heat map on at least one of the first and/or second disk.

### BRIEF DESCRIPTION OF THE DRAWINGS

FIG. 1 is a top view of a sleep wheel chart according to one embodiment of the disclosure.

FIG. 2 is a perspective exploded view of the wheel chart shown in FIGURE

FIG. 3 is a top view of a base shown in FIG. 2 and on which the rotatable disk layers of the wheel chart of FIG. 1 rotate;

FIG. 4 is a top view of a Circadian (C) Process disk layer of the sleep wheel chart of FIG. 2.

FIG. 5 is a top view of a Homeostatic (S) Process disk layer of the sleep wheel chart of FIG. 2.

FIGS. 6 and 7 are top views, respectively, of morning and evening light disk layers of the sleep wheel chart of FIG. 2.

### DETAILED DESCRIPTION

The present disclosure provides a wheel chart that includes a circadian graph with the use of polar coordinates to depict sleep and wake processes continuously throughout 24 hours. Although there are other minor processes also governing sleep pressures, the foundation of sleep physiology is based on two main processes. The first process, which is linked to the circadian rhythm, is known as Circadian (C) Process (or Process C). By way of background, Process C is based on a circadian-metabolic master clock located in the suprachiasmatic nucleus of the brain. It regulates many different body functions in a 24-hour cycle including the sleep/wake rhythm and hormonal rhythms. Process C brings about key phases of the sleep/wake rhythm including the afternoon sleepy period; the forbidden zone for sleep in the evening; and the pre-dawn phase of heavier sleep pressure. These phases affect the care of persons suffering from insomnia, circadian rhythm disorders, and even those without a sleep disorder. Disclosed herein is a device that conveys these aspects of sleep/wake tendencies.

The second process is known as Homeostatic (S) Process (or Process S—for “sleep”). This process links sleep pressure to the amount of time that a person has remained continuously awake and, in the cases of patients with sleep apnea, the many nights' Process S accumulation that the patient has not slept enough or with enough continuity. Ideally, the sleep pressure from Process S starts as fully discharged after a good night's sleep. One has little pressure from Process S in the early morning hours after awakening normally, but the level of pressure slowly builds throughout the day until it reaches a maximum when the person goes down for the night. A key feature of Process S is that it dissipates rapidly with any sleep. Thus, an afternoon nap can restart the Process S clock all over again. With the nap, the maximum pressure that is normally present at bedtime is pushed to later than normal, making it harder to go to sleep at the normal bedtime. The Process S physiology is regulated by a kind of “metabolic stop-watch” located in the brain. Additionally, its expression regulates the timed release of growth hormone in the early hours of the night and affects the release of other key daily hormone secretions.

The present disclosure relates to a visual aid or device in the form of a wheel chart that allows direct visualization of Processes C and S on sleep pressure when acting simultaneously, but independently. The independence of these two processes is not absolute, but the graph does present an overall true rendering of their general physiological independence from each other. One aspect of the wheel chart is that it is an educational tool: it provides a clearer explanation on the physiological reasons why a behavioral prescription (e.g., nap avoidance) would benefit a user. It also helps to illustrate how circadian body functions shift in response to light or melatonin.

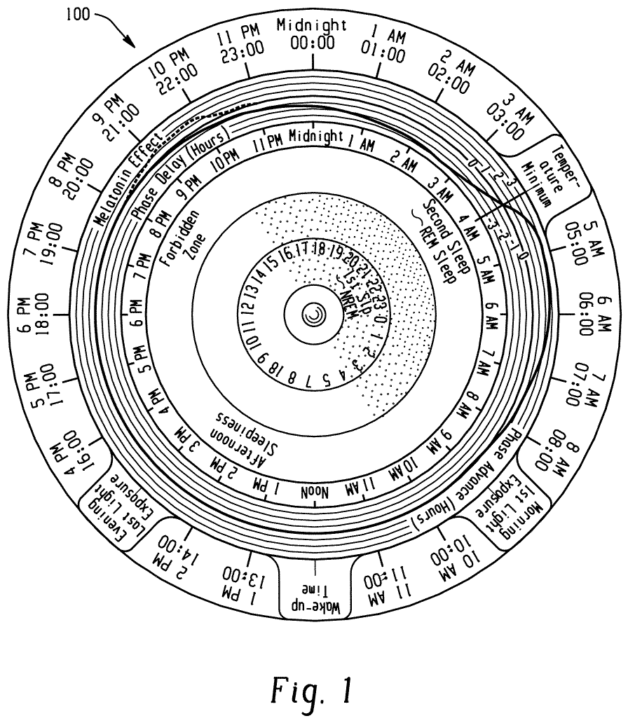

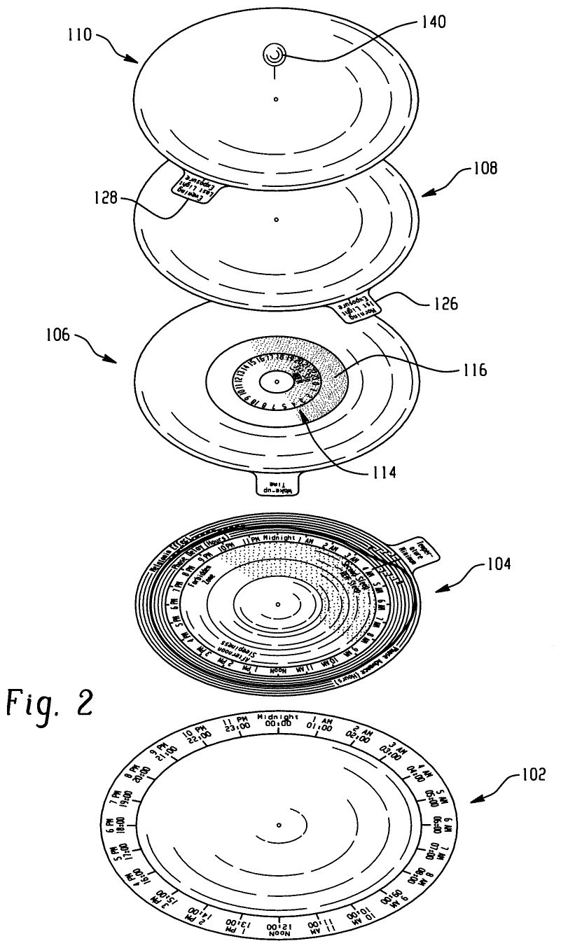

FIG. 1 shows a sleep wheel chart 100 according to one embodiment of the disclosure. A perspective exploded view of the wheel chart is also shown in FIG. 2. The wheel chart 100 is illustrated as a circular disk having a generally rigid base 102 and a series of moveable circular disks 104-110 positioned on a common axis 112. However, there is no limitation made herein to the shape or profile of the base 102. For example, base 102 can be a square body with a circular, fixed disk layer printed or displayed thereon.

There is no limitation made herein to the media on which the wheel chart is displayed. In one embodiment, the wheel chart can be printed or displayed on a physical media, such as a print media, for example paper or polystyrene. In these embodiments, the layers 104-110 can be secured to the base 102 at a common axis using a pin about which the layers can rotate. Instructions explaining the operation of the wheel chart can also be included on the base, but not within the circular graph portion. In another embodiment, the wheel chart can be displayed through an electronic media, such as a display device connected to a hardware processor.

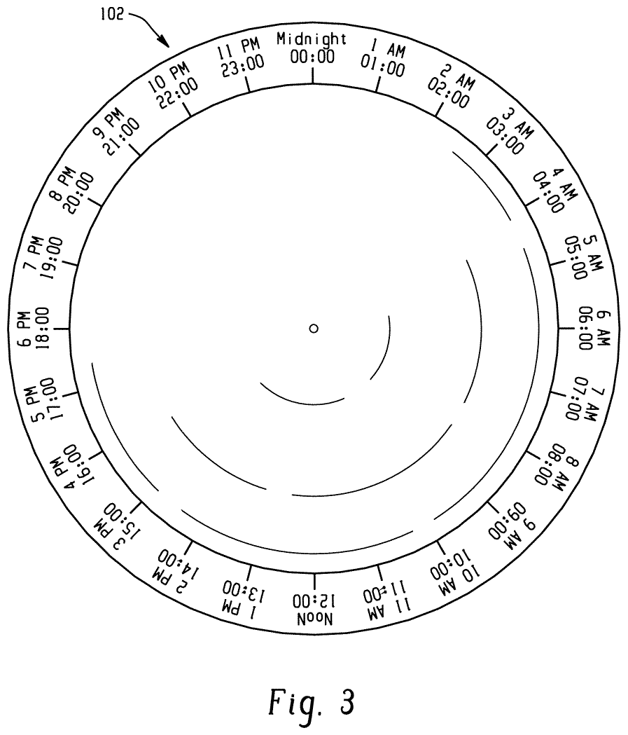

FIG. 3 is a top view of a circular, generally flat base shown in FIG. 2 and on which the rotatable disk layers of the wheel chart of FIG. 1 rotate. The base displays units of time along a perimeter of the base. In the illustrated embodiment, the units of time are in increments over a 24-hour period. The base displays units across a 24-hour environmental time. The disks for Process C and Process S that will overlay it will represent separate internal body clocks. FIG. 3 shows the units in hours so that every hour is displayed over the 24-hour cycle. However, there is no limitation made to the increments. The units of time can be in increments of 2-hours, 3-hours, half-hours, etc.

The Processes C and S are closely coordinated on days for most people on a regular daily schedule of sleep and wake. However, the Process S can be forced out of normal coordination with Process C for people on shift work. While substantial Process S pressure can slightly blunt circadian phase responses, these processes act independently of each other for the most part.

For this reason, at least two disk layers 104, 106 are provided on the wheel chart 100 to indicate to users how the processes contribute overt sleep pressures, even though they act independent of each other. The wheel chart 100 includes a first disk layer (hereinafter “disk” or “layer”) 104 representing a Circadian (C) Process and second disk 106 representing a Homeostatic (S) Process. The disks 104, 106 are selectively rotatable relative to each other to show how the C and S Processes interact to effect overall sleep pressure. In one embodiment, the disks 104, 106 (including, in some embodiments, disks 108 and 110) are translucent except where they graph their respective sleep pressures. In one embodiment, the sleep pressures are displayed as heat maps 112, 114. Depending on how the disks 104, 106 are rotated relative to each other, the heat maps 112, 114 can overlay at regions to give a visual representation of the combined effects of the two sleep pressures.

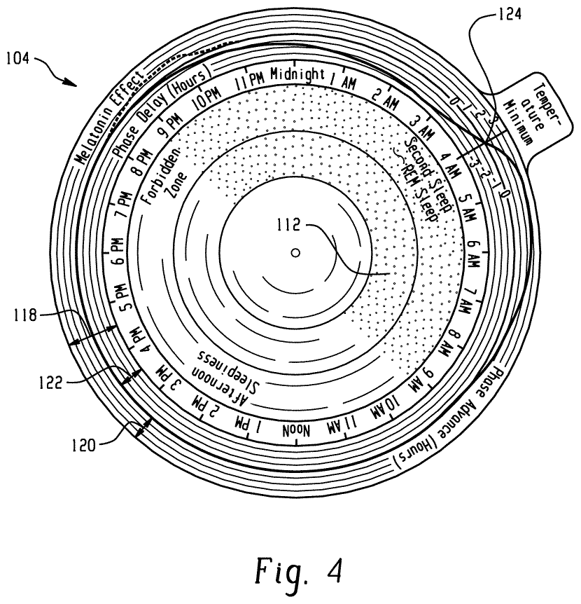

FIGS. 3 and 4 show that the heat maps are in proximate or similar regions (inside the disk—i.e., next to the axis) on their respective disks. Each heat map can employ shading or colors to show magnitude. In the contemplated embodiment, the shade corresponds to sleepiness, which is the force toward sleep that Process C and Process S impose on sleep. In the illustrated embodiments, darker shaded areas (e.g., 116) represent sleepiness and the clearer (less shaded areas relative to the darker areas) represent “pressure to be awake” or “awakeness”.

Now turning to FIG. 4. FIG. 4 is a top view of a Circadian (C) Process disk layer (also referred to herein as “Process C disk”) 104 of the sleep wheel chart of FIG. 2. The Process C disk 104 is divided (virtually or graphically) by several concentric rings. In one embodiment, at least two rings are provided. The Process C disk 104 includes the sleep pressure heat map 112 in a first concentric ring and a phase response curve (“PRC”) 118 in a second concentric ring. In the illustrated embodiment, the first (inner) concentric ring is closer to the central axis and the second (outer) concentric ring is farther from the axis.

In the first concentric ring, shading on the sleep pressure heat map 112 indicates a region of mild sleep. Depending on when the mild sleep pressure occurs (e.g., in the mid-afternoon), a user can identify when s/he would experience a time period known as the “forbidden zone” (e.g., 8 pm in the instance that the mild sleep pressure occurs in the mid-afternoon), when the Process C urges a person to feel or be more awake than at any other time in the 24-hour cycle.

In the second concentric ring, the PRC 118 is displayed as a line graph rendered on a circular axis. The PRC is located adjacent or close to the perimeter of the second disk 104 to reduce any psychological distortion of the polar display in the line graph.

The PRC is displayed in a way that depicts how the internal body clock can be phase shifted somewhat independently from the environmental time. It is graphed along the 24-hour period in units of shift in phase advance 120 or phase delay 122. The phase advance 120 and phase delay 122 are illustrated as two sub-concentric rings inside the first concentric ring to which the PRC curve belongs. In the illustrated embodiment, the PRC shows, by way of example, the melatonin effect in the PRC. Maximum possible phase shifting for a one-morning light therapy is known by those skilled in the art to be about three (3) hours. Therefore, the scale of the phase response curve (PRC) response goes from −3 to 3 hours. These are conveyed, in one embodiment, as circles within the PRC graphing domain.

In clinical practice, it is valuable to help patients not only understand the PRC responses to light in the morning, but also in the evening. While the morning light exposure will cause phase advances as represented in portion 120 of the PRC—provided it is not too early in relation to the PRC″s inflection point 124—the evening light exposure will cause phase delays as represented in portion 122. The inflection point 124 is illustrated in the PRC as a response to light around the time of the daily body temperature minimum. In the illustrated embodiment, by way of example only, this is shown around 4 am, when the body temperature reaches its minimum for most people.

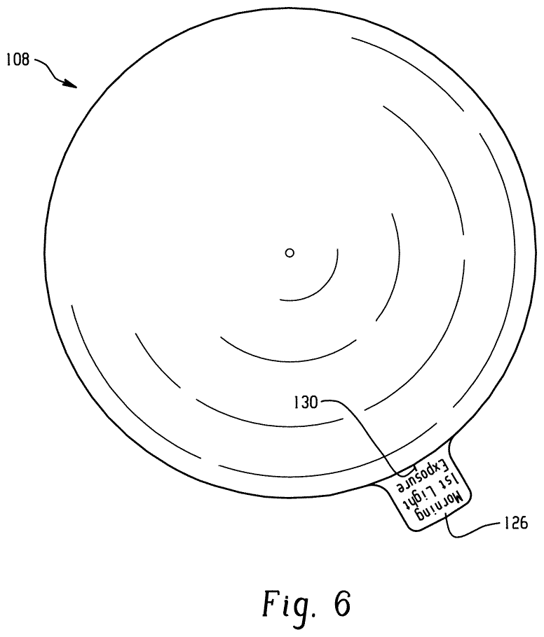

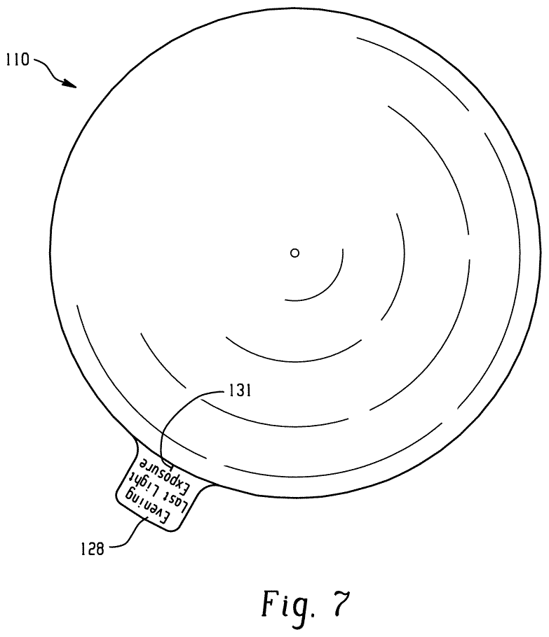

While the wheel chart 100 of the present disclosure considers three types of times: environmental time (in a solar day), Process C time, and Process S time, further embodiments can include the times when the processes induce phase shifts, wittingly or unwittingly, by light exposures. To illustrate this point, some embodiments include an additional two disks: a disk representing morning light exposure 108 (see FIG. 6) and a disk 110 representing evening light exposure (see FIG. 7). These times are conveyed on the PRC as two disks aid users in estimating the direction and scale of phase shifting that would occur with light exposures.

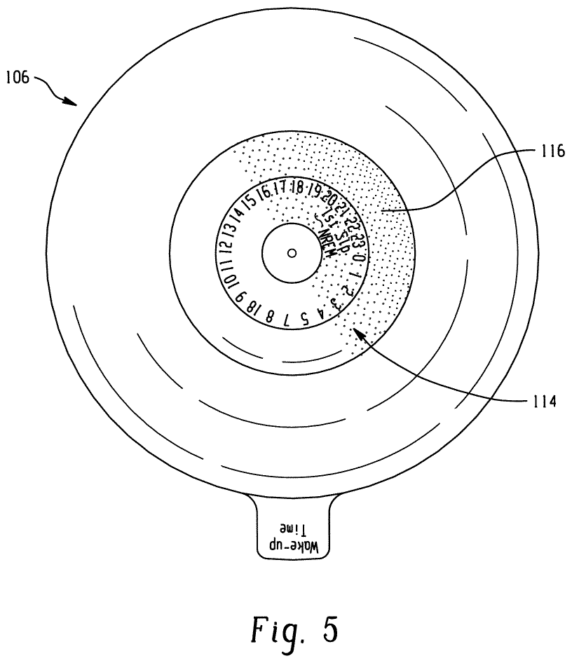

Continuing with FIG. 5, a top view of a homeostatic (S) process disk layer of the sleep wheel chart 100 is shown. Shading on the heat map 114 of the process S disk 106 shows the force toward sleepiness corresponding to shade. The shades increase as the time associated with the continuously awake period progresses.

Through the area where the heat maps 112, 114 overlap, the combined effects of both Processes S and C can be observed on the wheel chart 100. FIGS. 6 and 7 further show morning and evening, respectively, exposure timing tabs that may aid in estimating the direction and amount of phase response that may occur as a result of light exposure 126 (in the morning) or melatonin 128 (in the evening). To estimate a phase response, the tab 130, 132 representing light or melatonin exposure, respectively, is aligned with the time (shown on base 102) of the exposure. This will allow a user to read or calculate the number of hours that the phase will be advanced or delayed. A pointer 134 is moveable in the anti-clockwise or clockwise direction to estimate where the process C will change its inflection point.

In operation, as an illustrative example only, by noting the inflection point, a user can ascertain the affects that timing bright light therapy will have on delayed sleep phase syndrome. For example, by rotating the disks (using tabs 130) on the wheel chart, a user can observe the effects of bright light that is exposed too early for the body's clock. Similarly, the user can use the wheel chart to learn how to avoid (or what causes) jet lag symptoms by estimating when, during a series of nights before jet travel, to get light exposures, so to shift the body's circadian rhythm toward the destination environmental time even when a departure location's environment time remains unchanged.

The Process S disk 106 is selectively rotatable, but for most people it will not be moved from the start time for the build-up of sleep pressure in the morning (e.g., 6:00 am or 7:00 am for most people). However, the movability of this Process S disk will be especially helpful for educating the patient with insomnia who may falsely believe that an afternoon nap has no consequences for them. To note the effects of a nap on a user, the S Process disk 106 can be rotated to the time when the user wakes or is woken up from the nap. In response to this rotation, the wheel chart 100 can convey how the pressure from Process S was or will be delayed to later than the usual bedtime.

Therefore, by understanding that the greatest sleep pressure from Process C occurs in the hours before dawn, and is more associated with REM sleep, and deeper stages of NREM sleep occurs during the initial hours of nighttime sleep, the wheel chart or device of the present disclosure can illustrate why REM-related sleep disorders, such as REM behavior disorder, are more likely at this time of the night. And by understanding the greatest sleep pressure from Process S occurs earlier in the night, the device can be used to illustrate why arousal disorders, such as sleep walking, occur more frequently in this phase of sleep (known as “first sleep”).

Another potential operation of use of the sleep wheel is as an illustrative example of how a user can alter their wakeup time in order to shift their circadian rhythm to reduce or eliminate jet lag. This can be done by using the PRC as an aid for planning the change to the circadian rhythm in relation to a user's current environmental time to the time of their desired destination time. A user's current environmental time will relate to their usual wakeup time in their present time zone. If a user is traveling West of their time zone, they will need to delay their circadian rhythm to prevent jet lag, but if they are traveling East, they will need to advance their circadian rhythm to prevent jet lag.

The sleep wheel can assist a user in visualizing the processes of delaying or advancing their circadian rhythm by setting their current environmental wakeup time on the Process C wheel. The user will then determine what their goal wakeup time is in the new time zone is in relation to the current environmental wakeup time. The user will then determine whether they need to advance or delay their circadian rhythm in order to shift to their new wakeup time. The user will then use the PRC to determine what their new wakeup time must be in order to begin shifting their circadian rhythm. As discussed above the maximum recommended phase shifting goes from −3 to 3 hours, as is displayed on the PRC. Therefore, if a user is traveling to a location with a time change of greater than three hours, then the user will need to distribute their circadian rhythm shift over multiple nights in order to achieve the desired results. If this is necessary. then the user will determine their second new wakeup time by using the first new wakeup time as the starting time of the PRC, and then shifting the wakeup time as needed. This process will be repeated until the final new wakeup time is equal to the goal wakeup time.

Another aspect of the wheel chart of the present disclosure, in the form of a polar coordinate graph, is that it reduces the conceptual distortion that physiological processes are linear rather than circular. By presenting the sleep/wake processes in circular means, the chart allows a user to understand how the processes recycle back on themselves.

A further aspect of the present wheel chart is that it aids patients in understanding why behavioral prescriptions are broached by a medical professional, although the disclosed device is not contemplated to make precise predictions for individuals. These aids may also be used to teach students and medical professionals about circadian sleep phase disorders.

The exemplary embodiment has been described with reference to the preferred embodiments. Obviously, modifications and alterations will occur to others upon reading and understanding the preceding detailed description. It is intended that the exemplary embodiment be construed as including all such modifications and alterations insofar as they come within the scope of the appended claims or the equivalents thereof.

1. A wheel chart, comprising:
a series of moveable circular disks positioned on a common axis, the disks including a first disk representing a circadian process and a second disk representing a homeostatic process;
wherein the disks are rotatable relative to each other to display the effect of the interaction of the circadian process and the homeostatic process on overall sleep pressure by simulating a shift in circadian and homeostatic processes;
wherein the first disk displays information selected from the group consisting of:
a phase response curve;
a heat map of sleep pressures from the circadian process; and
a combination region for the co-representation of both the circadian process and the homeostatic process;
wherein the phase response curve is a line graph rendered on a circular axis along which units of time are displayed, wherein the units of time are in increments over a 24-hour period;
wherein the phase response curve is graphed along the 24-hour period in units of shift in phase advance or phase delay;
wherein the units of time are displayed on a fixed circular disk as a background on which the first disk overlays; and
wherein the first disk includes:
the phase response curve as an outer concentric ring along an edge of the first disk; and
a first heat map as an inner concentric ring relative to the phase response curve.

2-6. (canceled)

7. The wheel chart of claim 1, wherein the second disk includes a second heat map for the homeostatic process, wherein the second heat map translucently overlaps the first heat map in at least a region of the inner concentric ring.

8. The wheel chart of claim 7, wherein each disk includes a tab for rotating the disk around the common axis, each disk being independently rotatable around the common axis.

9. The wheel chart of claim 8, wherein the wheel chart is provided as one of print media and electronic format, the electronic format being displayed on an output device.

10. (canceled)

11. The wheel chart of claim 1 further comprising:
a disk representing morning light exposure;
a disk representing evening light exposure; or
a combination of them both.

12. A wheel chart graphing a circadian process and a homeostatic process on separate disks, the disks being rotatable relative to one another to display the physiological processes governing sleep and circadian rhythms, the circadian process being graphed as a first circular heat map and the homeostatic process being graphed as a second circular heat map, the first and second heat maps being rotatable relative to one another and at least partially translucently overlapping to display an effect of the combined circadian and homeostatic processes on sleep pressure.

13. The wheel chart of claim 12 further comprising the circadian process being graphed as a circular line graph and the homeostatic process being graphed as a circular heat map, the line graph and the heat map being rotatable relative to one another to display an effect of the combined circadian and homeostatic processes on sleep pressure.

14. The wheel chart of claim 13, wherein the circular line graph is a phase response curve.

15. The wheel chart of claim 14, wherein the phase response curve is a line graph rendered on a circular axis along which units of time are displayed, wherein the units of time are in increments over a 24-hour period.

16. The wheel chart of claim 14, wherein the phase response curve is graphed along the 24-hour period in units of shift in phase advance or phase delay.

17. (canceled)

18. The wheel chart of claim 12, wherein the wheel chart includes:
a phase response curve as an outer concentric ring on a first disk; and
at least one heat map as an inner concentric ring relative to the phase response curve, the at least one heat map on at least one of the first and/or a second disk.

19. The wheel chart of claim 12 further comprising a third disk representing morning light exposure and/or a fourth disk a disk representing evening light exposure.

20. A method of visualizing the shifting of a body's circadian rhythm:
providing a wheel chart, comprising a series of moveable circular disks positioned on a common axis, the disks including a first disk representing a circadian process and second disk representing a homeostatic process, wherein the disks are rotatable relative to each other to display the effect of the interaction of the circadian process and the homeostatic process on overall sleep pressure by how the circadian and homeostatic processes interact to effect sleep pressure, wherein the first disk displays a phase response curve;
selecting a goal wakeup time in relation to a current environmental time;
setting the circadian process disk to the current environmental time;
determining whether the goal wakeup time requires a delay or advance of the body's circadian rhythm;
using the phase response curve to establish a new wake up time to delay or advance the circadian rhythm;
setting the circadian process disk to the new wake up time; and
repeating the last two steps, if necessary, until the new wake up time is the goal wakeup time.
